(19) United States
(12) Patent Application Publication
(10) Pub. No.: US 2025/0127623 A1
(43) Pub. Date: Apr. 24, 2025

(54) CONTOURABLE BASEPLATE FOR A GLENOID

(71) Applicant: Zimmer, Inc., Warsaw, IN (US)

(72) Inventor: Cody Johnathon Holmes, Warsaw, IN (US)

(21) Appl. No.: 18/910,750

(22) Filed: Oct. 9, 2024

Related U.S. Application Data
(60) Provisional application No. 63/591,294, filed on Oct. 18, 2023.

(51) Int. Cl. A61F 2/40

(52) U.S. Cl. CPC A61F 2/4081

(57) ABSTRACT

A baseplate configured to mount on a surface of a glenoid and couple with a glenosphere for a reverse shoulder arthroplasty. The baseplate optionally comprising: a main body having a plurality of thru holes therein; and one or more wings coupled to the main body and extending outward therefrom, wherein the one or more wings include, at least one thru hole therein, and wherein the one or more wings are configured to be moveable relative to the main body to interface with the surface of the glenoid.

### CLAIM OF PRIORITY

This application claims the benefit of U.S. Provisional Patent Application Ser. No. 63/591,294, filed on Oct. 18, 2023, the benefit of priority of which is claimed hereby, and which is incorporated by reference herein in its entirety.

### TECHNICAL FIELD

The present disclosure relates to orthopedic implants and methods for implanting such implants into the body of a patient. In particular, the present disclosure relates to orthopedic implant for the glenoid of the shoulder, related features, components and methods of implantation.

### BACKGROUND

The shoulder joint includes a humerus bone and a scapula bone, which cooperate to provide range of motion of the humerus relative to the scapula during movement of a human arm. Specifically, a proximal end of the humerus including a humeral head is disposed adjacent to a glenoid fossa (simply termed “glenoid” herein) of the scapula and is permitted to move relative to the glenoid fossa to provide a range of motion to the humerus relative to the scapula.

Joint replacement surgery such as a partial or total shoulder arthroplasty may be required or desired when the shoulder joint causes pain during use or is otherwise damaged. For example, the shoulder joint may be damaged due to osteoarthritis, whereby progressive wearing away of cartilage results in bare bone being exposed within the shoulder joint. Shoulder joint reconstruction may require repairing a defect in a shoulder joint such as a void in the glenoid resulting from severe wear. Under such circumstances, it is often necessary or desirable to undergo a partial or total shoulder arthroplasty in order to relieve pain and increase the range of motion of the humerus by rebuilding portions of the shoulder joint.

Reverse-type shoulder implant systems have been developed in which the conventional ball-and-socket configuration that replicates the natural anatomy of the shoulder is reversed, such that a concave recessed articulating component is provided at the proximal end of the humeral component that articulates against a convex portion of a glenosphere of a glenoid component. More specifically, a reverse total shoulder arthroplasty (rTSA), can involve implanting a glenosphere on a patient's glenoid and a humeral stem into the humerus. The humeral stem can have a bearing that interacts with the glenosphere to restore normal shoulder function. In some cases, a baseplate can be attached to the patient's glenoid to provide for an anchor to the bone. The baseplate can engage with the glenosphere to secure the glenosphere relative to the glenoid.

In some reverse-type shoulder implant procedures, a patient's glenoid may also have a defect. This can require eccentric reaming or some other suitable preparation of the glenoid for the correction of the defect. This can result in a situation in which a certain portion of the patient's glenoid has more bone removed relative to other portions of the glenoid. In such cases, an augment might be used with a baseplate to ensure proper support of the baseplate by the patient's bone. Likewise, one or more screws might extend through the baseplate and/or the augment to secure both components to the glenoid. More healthy bone than may be desired may be removed to accommodate the augment design. Additionally, proper screw trajectories must be utilized by the surgeon to provide adequate fixation of the baseplate to the bone.

### OVERVIEW

The present inventor has recognized, among other things, that problems to be solved in preparing a glenoid of a shoulder joint to receive a baseplate include the difficulty of selecting a proper augment to address any bone defect(s), preparing the bone to receive the augment(s) selected and subsequently determining and achieving a proper screw fixation orientation(s). The present inventor recognizes that some amount of healthy bone may be removed unnecessarily to accommodate the augment. Loss of healthy bone during a procedure is undesirable. Additionally, addressing the defect(s), preparing the bone to receive the augment(s), and orienting screws properly for fixation adds to surgical complexity and procedure time.

The present subject matter can provide solutions to these and other problems, such as by providing a baseplate that can be contoured in vivo to mate with the surface of the glenoid. This conforming baseplate can address defects in the glenoid without the need for augments or removal of bone. Thus, the baseplate can be mounted to the glenoid in a bone preserving manner. The baseplate designs of the present application can include one or more wings that can be flexible or otherwise deflectable to interface with the surface of the glenoid. The present baseplate designs can eliminate the need for augments and bone forming such that surgical time and complexity can be reduced.

To better illustrate the apparatuses, systems and methods disclosed herein, a non- limiting list of examples is provided here:

Example 1 is a baseplate configured to mount on a surface of a glenoid and couple with a glenosphere for a reverse shoulder arthroplasty, optionally comprising: a main body having a plurality of thru holes therein; and one or more wings coupled to the main body and extending outward therefrom, wherein the one or more wings include, at least one thru hole therein, and wherein the one or more wings are configured to be moveable relative to the main body to interface with the surface of the glenoid.

In Example 2, the subject matter of Example 1 optionally includes, a depth stop coupled to the one or more wings, wherein the depth stop includes a thru hole arranged generally coaxial with the at least one thru hole, wherein the depth stop is positioned at least one of adjacent to or at least partially within at least one of the plurality of thru holes of the main body.

In Example 3, the subject matter of Example 2 optionally includes, wherein the depth stop is one of: integral with the one or more wings or an insert separate from the one or more wings.

In Example 4, the subject matter of Examples 2-3 optionally includes, wherein, when the one or more wings are in an undeflected state, the depth stop includes a proximal surface that is acutely angled relative to a centerline axis of the thru hole.

In Example 5, the subject matter of Example 4 optionally includes, wherein the main body has a centerline axis, and wherein an outer radial portion of the depth stop from the centerline axis has an increased depth as compared with an inner radial portion of the depth stop from the centerline axis.

In Example 6, the subject matter of Examples 4-5 optionally includes, one or more fixation elements received by one or more of the plurality of thru holes of the main body and the at least one thru hole of the one or more wings, wherein a head of the one or more fixation elements engages the proximal surface of the depth stop to deflect the one or more wings relative to the main body to interface with the surface of the glenoid.

In Example 7, the subject matter of Examples 1-6 optionally includes, one or more fixation elements received by one or more of the plurality of thru holes of the main body and the at least one thru hole of the one or more wings, wherein the one or more fixation elements are configured to force the one or more wings against the surface of the glenoid.

In Example 8, the subject matter of Examples 1-7 optionally includes, wherein the one or more wings include between one and four wings, inclusive, and wherein each of the between one and four wings includes the at least one thru hole.

In Example 9, the subject matter of Examples 1-8 optionally includes, a peg coupled to and extending from the main body, wherein one of the plurality of thru holes of the main body additionally passes through the peg, and wherein the one or more wings are positioned around the peg.

In Example 10, the subject matter of Example 9 optionally includes, wherein the peg and the one or more wings are at least partially formed by a porous material configured to facilitate tissue ingrowth.

Example 11 is an orthopedic system for a reverse shoulder arthroplasty, optionally comprising: a one or more fixation elements; and a baseplate configured to mount on a surface of a glenoid and couple with a glenosphere, the baseplate comprising: a main body having a plurality of thru holes therein, one or more wings coupled to the main body and extending outward therefrom, wherein the one or more wings include, at least one thru hole therein, and a depth stop coupled to each of the one or more wings, wherein the depth stop includes a thru hole with a centerline axis generally aligned with a centerline axis of the at least one thru hole, wherein the depth stop is positioned at least one of adjacent to or at least partially within at least one of the plurality of thru holes of the main body, wherein a proximal surface of the depth stop is configured to be engaged by a head of the one or more fixation elements to deflect the one or more wings relative to the main body to interface with the surface of the glenoid.

In Example 12, the subject matter of Example 11 optionally includes, wherein the depth stop is one of: integral with the one or more wings or an insert separate from the one or more wings.

In Example 13, the subject matter of Examples 11-12 optionally includes, wherein, when the one or more wings are in an undeflected state, the depth stop includes a proximal surface that is acutely angled relative to a centerline axis of the thru hole.

In Example 14, the subject matter of Examples 11-13 optionally includes, wherein the one or more wings include between one and four wings, inclusive, and wherein each of the between one and four wings includes the at least one thru hole.

Example 15 is a method of implanting a baseplate to a glenoid during a reverse shoulder arthroplasty, the method optionally comprising: preparing the glenoid to receive the baseplate; positioning the baseplate on a surface of the glenoid; deflecting a portion of the baseplate to generally conform with the surface of the glenoid by engaging one or more fixation elements with the portion of the baseplate; and passing at least a portion of the one or more fixation elements into the glenoid.

In Example 16, the subject matter of Example 15 optionally includes, wherein deflecting the portion of the baseplate includes engaging a depth stop with a head of the one or more fixation elements.

In Example 17, the subject matter of Example 16 optionally includes, coupling the depth stop to the portion of the baseplate sequent to deflecting the portion of the baseplate.

In Example 18, the subject matter of Examples 15-17 optionally includes, wherein deflecting the portion of the baseplate includes selectively deflecting two or more portions of the baseplate.

In Example 19, the subject matter of Example 18 optionally includes, deflecting the two or more portions of the baseplate to a different depth from one another.

In Example 20, the subject matter of Examples 15-19 optionally includes, anchoring the baseplate to the glenoid with a single fixation element partially received by a centrally positioned peg of the baseplate.

Example 21 is at least one machine-readable medium optionally including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

In Example 25, the apparatuses, methods or systems of any one or any combination of Examples 1-24 can optionally be configured such that all elements or options recited are available to use or select from, can stand on its own, or can be combined in various permutations or combinations with one or more of the other examples.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a baseplate having a plurality of moveable wings according to an example of the present application.

FIG. 1A is a side view of the baseplate of FIG. 1.

FIG. 1B is a proximal side view of the baseplate of FIG. 1.

FIG. 1C is a first cross-sectional view of the baseplate of FIG. 1B.

FIG. 1D is a second cross-sectional view of the baseplate of FIG. 1B.

FIG. 2A is side view of the baseplate of FIGS. 1-1D implanted on a glenoid of a patient with a plurality of fixation elements received by the baseplate and the wings of the baseplate deflected to conform to a surface of the glenoid according to an example of the present application.

FIG. 2B is a cross-sectional view of the baseplate and some of the plurality of fixation elements of FIG. 2A implanted on the glenoid.

FIG. 3A is a side view of a baseplate with a single wing according to another example of the present application.

FIG. 3B is a cross-sectional view of the baseplate of FIG. 3A.

FIG. 4A is a side view of the baseplate of FIG. 3A implanted on a glenoid of a patient with a plurality of fixation elements received by the baseplate and the single wing of the baseplate deflected to conform to a surface of the glenoid according to an example of the present application.

FIG. 4B is an enlarged cross-sectional view of the single wing and one of the fixation elements of FIG. 4A.

FIG. 5 is a perspective view of a baseplate having a plurality of moveable wings according to another example of the present application.

FIG. 5A is a side view of the baseplate of FIG. 5.

FIG. 5B is a proximal side view of the baseplate of FIG. 5.

FIG. 5C is a distal side view of the baseplate of FIG. 5.

FIG. 5D is a cross-sectional view of the baseplate of FIG. 5B.

FIG. 6 is a flow diagram illustrating a method of implanting a baseplate such as an example of the present application.

### DETAILED DESCRIPTION

Examples of the present application are described in reference to the shoulder joint of a patient. However, it should be recognized that the techniques, methods, systems and prostheses described can be applicable to any anatomical feature including any joint of the patient. The anatomical feature can also include a non-joint portion of a bone or other anatomy of the patient (e.g., soft tissue) in some cases.

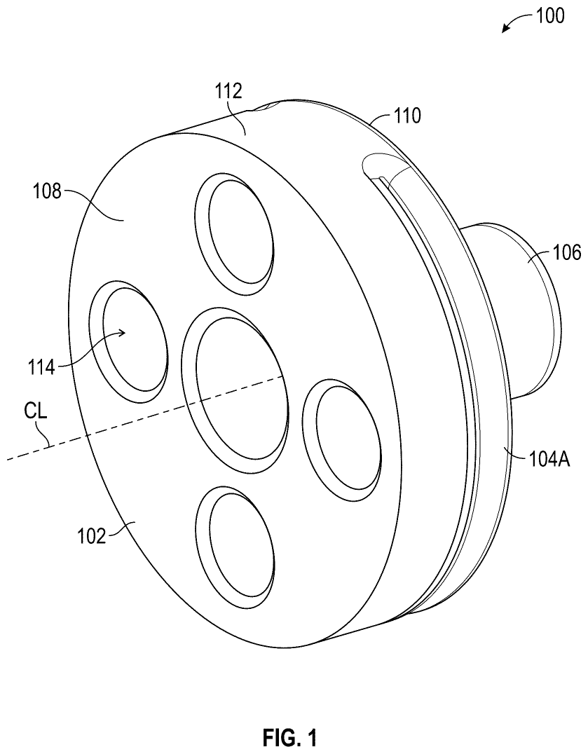

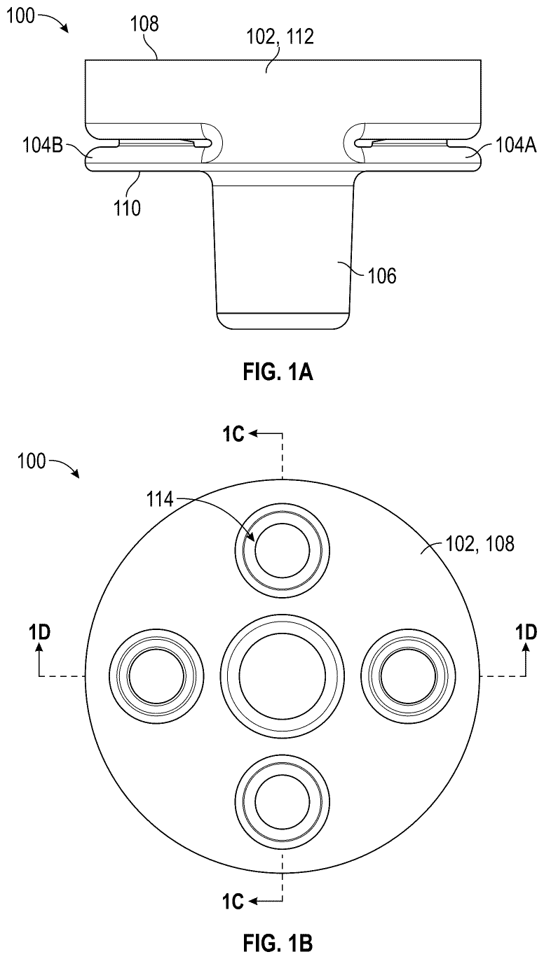

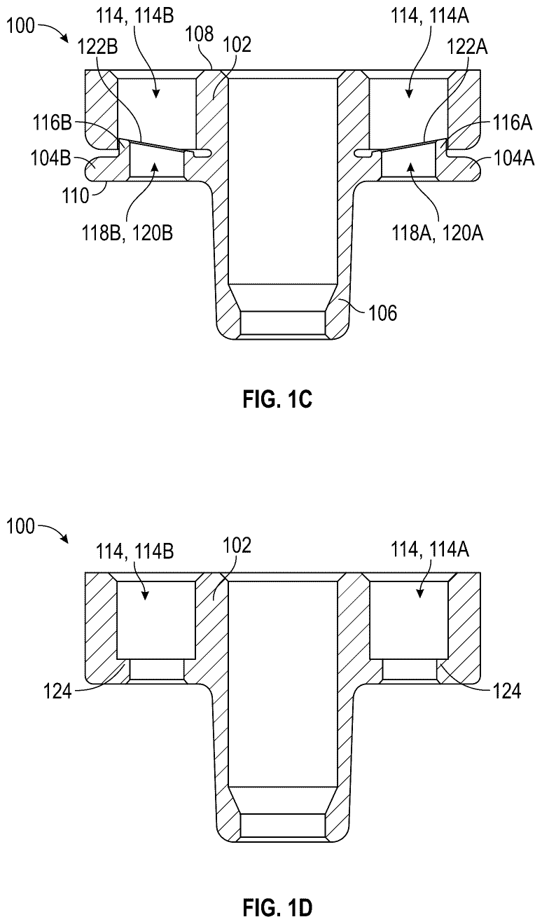

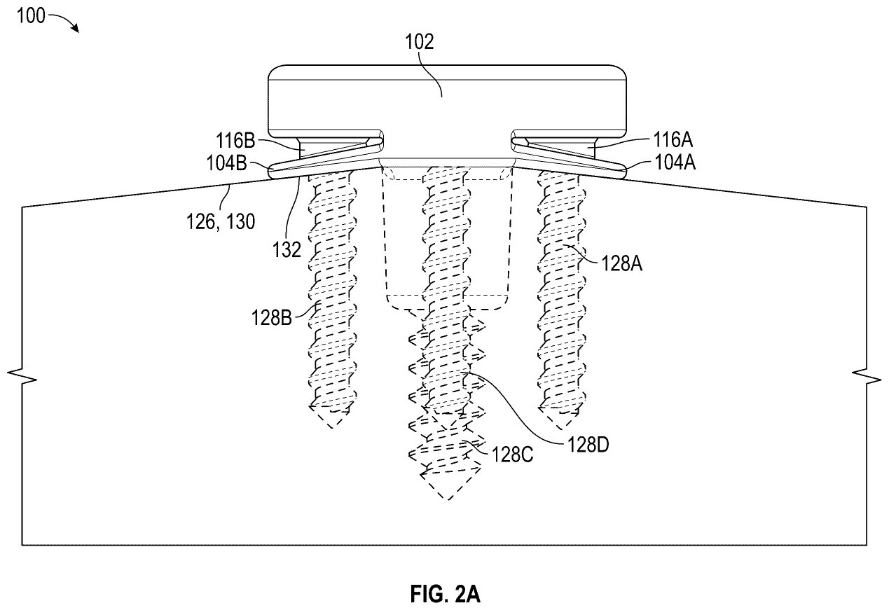

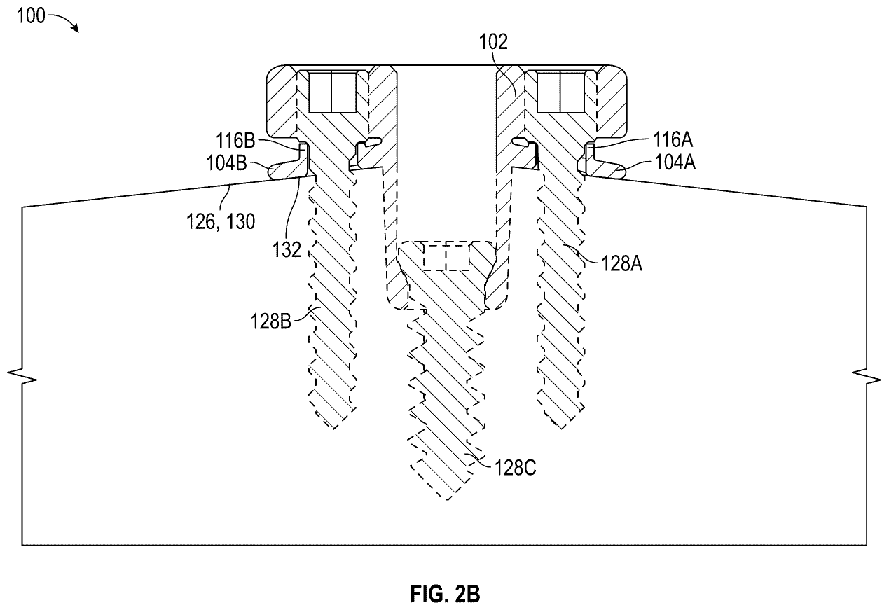

FIG. 1 is a perspective view of a baseplate 100 of the present disclosure configured to conform with a surface of a glenoid, such as shown in FIGS. 2A and 2B, for example. As shown in FIGS. 1-1D, the baseplate 100 can include a main body 102, a first wing 104A, a second wing 104B (not shown in FIGS. 1 and 1C), and a peg 106 (now shown in FIG. 1C). As shown in various of FIGS. 1-1D, the main body 102 can include a centerline axis CL, a proximal surface 108, a distal surface 110, a periphery 112 and a plurality of thru holes 114.

The first wing 104A and the second wing 104B connect to the main body 102 at an inner radial portion and can extend generally radially outward from the main body 102 relative to the centerline axis CL thereof. The outer edge of the first wing 104A and the second wing 104B can be generally co-extensive with the periphery 112 of the main body 102. However, alternative examples contemplate the first wing 104A and the second wing 104B can extend relatively further radially outward than the periphery 112. The first wing 104A and the second wing 104B can be positioned adjacent the peg 106. The peg 106 can extend generally distally from the main body 102. The first wing 104A and the second wing 104B can at least partially form the distal surface 110 in addition to the main body 102.

Although illustrated as generally cylindrically shaped in FIG. 1, the main body 102 can have a different shape in cross-section such as oval or non-uniform, for example. The depth (thickness) of the main body 102 can vary as desired. The proximal surface 108 can be generally opposed by the distal surface 110. The peg 106 and/or the distal surface 110, formed by parts of the main body 102, the first wing 104A and the second wing 104B can be at least partially formed by a porous material.

The porous material can have a substrate that incorporates one or more of a variety of biocompatible metals such as aluminum, titanium, a titanium alloy, cobalt chromium, cobalt chromium molybdenum, tantalum, or a tantalum alloy. According to an exemplary embodiment of the present disclosure, the substrate may be a Ti—6Al—4V alloy, such as Tivanium®, OsseoTi® or other porous which is available from Zimmer Biomet, Inc., of Warsaw, Ind. The porous material can have a highly porous metal structure that can incorporate one or more of a variety of biocompatible metals. Such structures are particularly suited for contacting bone and soft tissue, and in this regard, can be useful as a bone substitute and as cell and tissue receptive material, for example, by allowing tissue to grow into the porous structure over time to enhance fixation (i.e., osseointegration) between the structure and surrounding bodily structures. According to certain embodiments of the present disclosure, an open porous metal structure may have a porosity as low as 55%, 65%, or 75% or as high as 80%, 85%, or 90%, or within any range defined between any pair of the foregoing values. An example of an open porous metal structure is produced using Trabecular Metal™ Technology available from Zimmer Biomet, Inc. Such a material may be formed from a reticulated vitreous carbon foam substrate which is infiltrated and coated with a biocompatible metal, such as tantalum, by a chemical vapor deposition (“CVD”) process in the manner disclosed in detail in U.S. Pat. No. 5,282,861 and in Levine, B. R., et al., “Experimental and Clinical Performance of Porous Tantalum in Orthopedic Surgery”, Biomaterials 27 (2006) 4671-4681, the disclosures of which are expressly incorporated herein by reference. In addition to tantalum, other biocompatible metals may also be used in the formation of a highly porous metal structure such as aluminum, titanium, a titanium alloy, cobalt chromium, cobalt chromium molybdenum, tantalum, a tantalum alloy, niobium, or alloys of tantalum and niobium with one another or with other metals. It is also within the scope of the present disclosure for a porous metal structure to be in the form of a fiber metal pad or a sintered metal layer, such as a Cancellous-Structured Titanium™ (CSTi™) layer. CSTi™ porous layers are manufactured by Zimmer Biomet, Inc.

The highly porous metal structure will include a large plurality of metallic ligaments defining open voids (i.e., pores) or channels therebetween. The open spaces between the ligaments form a matrix of continuous channels having few or no dead ends, such that growth of soft tissue and/or bone through open porous metal is substantially uninhibited. Thus, the open porous metal may provide a lightweight, strong porous structure which is substantially uniform and consistent in composition, and provides a matrix (e.g., closely resembling the structure of natural cancellous bone) into which soft tissue and bone may grow to provide fixation of the implant to surrounding bodily structures. According to some aspects of the present disclosure, exterior surfaces of an open porous metal structure can feature terminating ends of the above-described ligaments. Such terminating ends can be referred to as struts, and they can generate a high coefficient of friction along an exposed porous metal surface. Such features can impart an enhanced affixation ability to an exposed porous metal surface for adhering to bone and soft tissue. Also, when such highly porous metal structures are coupled to an underlying substrate, a small percentage of the substrate may be in direct contact with the ligaments of the highly porous structure, for example, approximately 15%, 20%, or 25%, of the surface area of the substrate may be in direct contact with the ligaments of the highly porous structure.

An open porous metal structure may also be fabricated such that it comprises a variety of densities in order to selectively tailor the structure for particular orthopedic applications. In particular, as discussed in the above-incorporated U.S. Pat. No. 5,282,861, an open porous metal structure may be fabricated to virtually any desired density, porosity, and pore size (e.g., pore diameter), and can thus be matched with the surrounding natural tissue in order to provide an improved matrix for facilitating tissue ingrowth and mineralization. According to certain embodiments, an open porous metal structure may be fabricated to have a substantially uniform porosity, density, and/or void (pore) size throughout, or to comprise at least one of pore size, porosity, and/or density being varied within the structure. For example, an open porous metal structure may have a different pore size and/or porosity at different regions, layers, and surfaces of the structure. The ability to selectively tailor the structural properties of the open porous metal, for example, enables tailoring of the structure for distributing stress loads throughout the surrounding tissue and promoting specific tissue ingrown within the open porous metal.

An open porous metal structure may comprise an open cell polyurethane foam substrate coated with Ti—6Al—4V alloy using a low temperature arc vapor deposition process. Ti—6Al—4V beads may then be sintered to the surface of the Ti—6Al—4V-coated polyurethane foam substrate. Additionally, another embodiment of an open porous metal structure may comprise a metal substrate combined with a Ti—6Al—4V powder and a ceramic material, which is sintered under heat and pressure. The ceramic particles may thereafter be removed leaving voids, or pores, in the substrate. An open porous metal structure may also comprise a Ti—6Al—4V powder which has been suspended in a liquid and infiltrated and coated on the surface of a polyurethane substrate. The Ti—6Al—4V coating may then be sintered to form a porous metal structure mimicking the polyurethane foam substrate. Further, another embodiment of an open porous metal structure may comprise a porous metal substrate having particles, comprising altered geometries, which are sintered to a plurality of outer layers of the metal substrate. Additionally, an open porous metal structure may be fabricated according to electron beam melting (EBM) and/or laser engineered net shaping (LENS). For example, with EBM, metallic layers (comprising one or more of the biomaterials, alloys, and substrates disclosed herein) may be coated (layer by layer) on an open cell substrate using an electron beam in a vacuum. Similarly, with LENS, metallic powder (such as a titanium powder, for example) may be deposited and coated on an open cell substrate by creating a molten pool (from a metallic powder) using a focused, high-powered laser beam.

As shown in FIGS. 1C-1D, the plurality of thru holes 114 can pass through the main body 102 from the proximal surface 108 to the distal surface 110 portions formed by the main body 102. One of the plurality of thru holes 114 can be centrally located so as to generally aligned with the centerline axis CL of the main body 102. This one of the plurality of thru holes 114 can extend through the peg 106 in addition to the main body 102 and can be tapered in a manner known in the art. A first 114A of the plurality of thru holes 114 can extend through the main body 102 to the first wing 104A. A second 114B of the plurality of thru holes 114 can extend through the main body 102 to the second wing 104B. The plurality of thru holes 114 can be threaded or otherwise configured to secure with fixation elements such as bone screws to mount the baseplate 100 to the glenoid. At least some of the plurality of thru holes 114 can be configured with counterbores to receive a head of respective of the fixation elements as further illustrated in the present application.

The first wing 104A and the second wing 104B can be formed of suitable deflectable or deformable materials that can move relative to the main body 102 under an applied load from the fixation element(s). Deflection or deformation can be temporary (under applied force from the fixation element(s)) or permanent. As such, the first wing 104A and the second wing 104B can be formed from a metal, metal alloy(s), polymer, composite or other suitable material. The first wing 104A and the second wing 104B can be sufficiently thin along a connection to the main body 102 so as to be moveable under the applied load to generally conform with the surface of the glenoid as illustrated subsequently. Although the first wing 104A and the second wing 104B are shown as relatively uniform in depth (thickness) along a radial extent thereof, other examples contemplate a varying thickness or different distinct portions having different thicknesses. The distal surface 110 can be substantially uniform in depth as shown in FIGS. 1A, 1C and 1D. However, according to further examples, the distal surface 110 formed by the first wing 104A and/or the second wing 104B can differ in depth from the portion of the distal surface 110 formed by the main body 102.

FIG. 1C shows an example where a majority of the first wing 104A and the second wing 104B is spaced a distance from the main body 102 as measured in an axial direction relative to the centerline axis CL of the main body 102. Thus, the first wing 104A and the second wing 104B can cantilever from the main body 102 and/or peg 106.

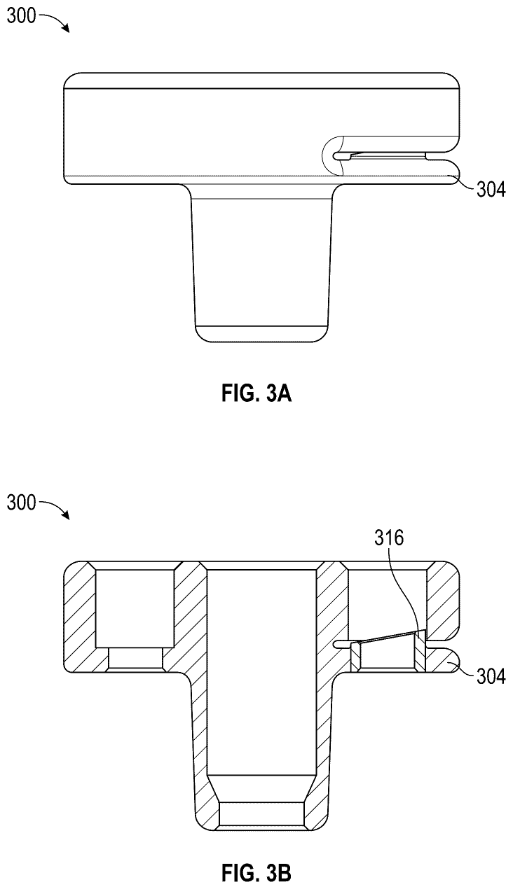

As shown in FIG. 1C, the baseplate 100 includes a first depth stop 116A and a second depth stop 116B. The first depth stop 116A can couple with the first wing 104A. The second depth stop 116B can couple with the second wing 104B. The example of FIG. 1C shows the first depth stop 116A integral with the first wing 104A and the second depth stop 116B integral with the second wind 104B. Thus, the first depth stop 116A can be a formed part of the first wing 104A and is a feature thereof. The second depth stop 116B can be a formed part of the second wing 104B and is a feature thereof. However, other embodiments including those of FIGS. 3A-5D contemplate the depth stop can be a separate component from the wing. Thus, the depth stop can be an insert such as a sleeve or collar that is coupled to the wing, for example.

As shown in FIG. 1C, the first wing 104A can be positioned adjacent the thru hole 114A of the main body 102. In particular, the first depth stop 116A can be positioned at least one of adjacent to or at least partially within the thru hole 114A of the main body 102. The second wing 104B can be positioned adjacent the thru hole 114B of the main body 102. The second depth stop 116B can be positioned at least one of adjacent to or at least partially within the thru hole 114B of the main body 102. The first wing 104A can include a thru hole 118A, which can be generally aligned with an axis of the thru hole 114A. However, the thru hole 118A can have a generally smaller diameter than the thru hole 114A. The first depth stop 116A includes a thru hole 120A. The thru hole 120A can aligned so as to be arranged generally coaxial with the thru hole 118A. The configuration of the thru holes 114A, 118A and 120A allows for passage of portions of a fixation element through the baseplate 100 into the glenoid but can allow for a head or other portions of the fixation element to engage with the first wing 104A and/or the first depth stop 116A.

The second wing 104B can include a thru hole 118B, which can be generally aligned with an axis of the thru hole 114B. However, the thru hole 118B can have a generally smaller diameter than the thru hole 114B. The second depth stop 116B can include a thru hole 120B. The thru hole 120B can aligned so as to be arranged generally coaxial with the thru hole 118B. The configuration of the thru holes 114B, 118B and 120B allows for passage of portions of a fixation element through the baseplate 100 into the glenoid but can allow for a head or other portions of the fixation element to engage with the second wing 104B and/or the second depth stop 116B.

The first depth stop 116A can include a proximal surface 122A that is acutely angled relative to a centerline axis of the thru hole 120A when the first wing 104A is in an undeflected state as shown in FIG. 1C. Similarly, the second depth stop 116B can include a proximal surface 122B that is acutely angled relative to a centerline axis of the thru hole 120B when the second wing 104B is in the undeflected state as shown in FIG. 1C. This configuration can allow the proximal surfaces 122A, 122B to be canted or angled relative to the proximal surface 108 and the distal surface 110 when the first and second wings 104A and 104B are undeflected, for example. Put another way, an outer radial portion of the first depth stop 116A from the centerline axis CL of the main body 102 has an increased depth (thickness) as compared with an inner radial portion of the first depth stop 116A from the centerline axis CL. Similarly, an outer radial portion of the second depth stop 116B from the centerline axis CL of the main body 102 has an increased depth (thickness) as compared with an inner radial portion of the second depth stop 116B from the centerline axis CL.

FIG. 1D shows a second cross-section of the baseplate 100 where the plurality of thru holes 114 through the main body 102 include a counterbore portion 124 to provide a stop for the fixation elements.

FIGS. 2A and 2B show the baseplate 100 of FIGS. 1-1D implanted on a glenoid 126 of a patient with a plurality of fixation elements 128A, 128B, 128C and 128D (shown in FIG. 2A only) received by the main body 102 and the first and second wings 104A and 104B and with the first and second wings 104A and 104B of the baseplate 100 moved to interface with a surface 130 of the glenoid 126 having two deformities 132. These deformities 132 can be recesses in the glenoid 126, for example. As shown, the fixation element 128A engages the first depth stop 116A and forces the first wing 104A to move into the deflected position against the surface 130 as shown in FIGS. 2A and 2B. The fixation element 128B engages the second depth stop 116B and forces the second wing 104B to move into the deflected position against the surface 130 as shown in FIGS. 2A and 2B. Although the first wing 104A and the second wing 104B are shown in FIGS. 2A and 2B as having a relatively a same amount of deflection, other examples contemplate the first wing 104A can have an amount of deflection that differs from that of the second wing 104B based upon a differing depth of the deformities 132, for example.

FIGS. 2A and 2B show the fixation elements 128A, 128B, 128C and 128D (shown in FIG. 2A) as bone screws. However, other examples contemplate the fixation elements as k-wire, nails, rods or other suitable fixation components as known in the art. FIG. 2B shows the fixation element 128A received by the thru holes 114A, 118A and 120A (FIG. 1C). A head of the fixation element 128A can be threaded down on corresponding thread of the thru hole 114A (FIG. 1C). A distal end of the head of the fixation element 128A engages the proximal surface 122A (FIG. 1C) of the first depth stop 116A to deflect the first wing 104A relative to the main body 102 to interface with the surface 130 of the glenoid 126. The fixation element 128B is received by the thru holes 114B, 118B and 120B (FIG. 1C). A head of the fixation element 128B can be threaded down on corresponding thread of the thru hole 114B (FIG. 1C). A distal portion of the head of the fixation element 128B engages the proximal surface 122B (FIG. 1C) of the second depth stop 116B to deflect the second wing 104B relative to the main body 102 to interface with the surface 130 of the glenoid 126.

It should be noted that although the present application shows the first wing 104A and the second wing 104B deflected distally to conform with the surface 130 of the glenoid 126, it is contemplated that the first wing 104A and/or 104B could be deflected slightly proximally to accommodate a projecting deformity of the glenoid 126.

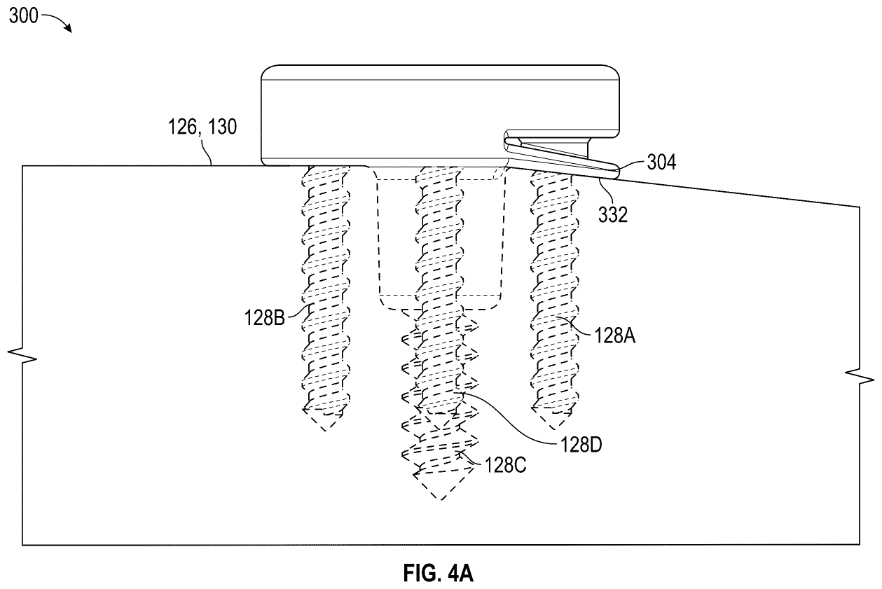

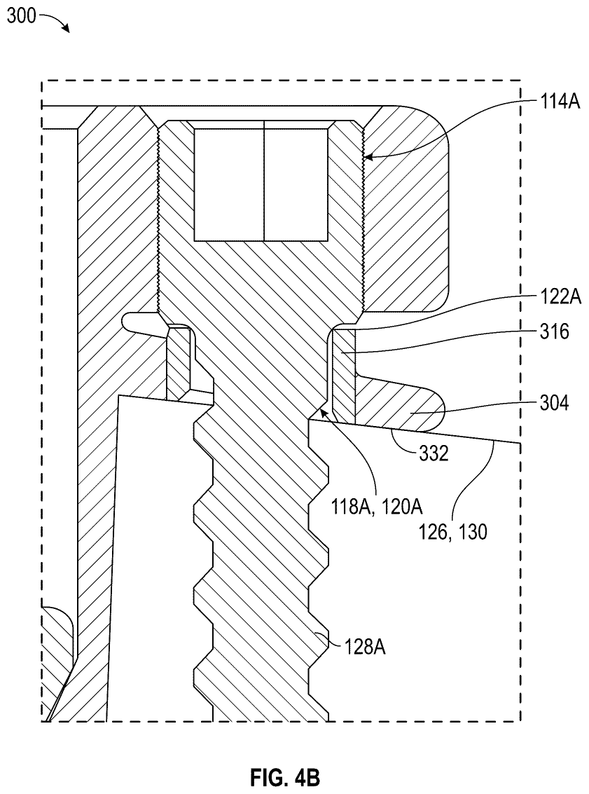

FIGS. 3A and 3B show a baseplate 300 according to an alternative example. The baseplate 300 differs from the baseplate 100 of the previous FIGURES in that it has only a single wing 304 that is moveable to interface with a surface 130 of the glenoid 126 having only a single deformity 332 (see FIGS. 4A and 4B). As shown in FIG. 3B, the baseplate 300 additionally differs from the baseplate 100 in that a depth stop 316 is a separate component from the single wing 304. The depth stop 316 can be threaded or otherwise connected to single wing 304, and thus, is an insert.

FIGS. 4A and 4B show the baseplate 300 implanted on the glenoid 126 of the patient with the plurality of fixation elements 128A, 128B, 128C and 128D (shown in FIG. 4A only) received by the baseplate 300 and the single wing 304 of the baseplate 300 moved to interface with the surface 130 of the glenoid 126 having the deformity 332. The enlarged cross-sectional view of FIG. 4B shows the fixation element 128A is received by the thru holes 114A, 118A and 120A. A head of the fixation element 128A can be threaded down on corresponding thread of the thru hole 114A. A distal portion of the head of the fixation element 128A engages the proximal surface 122A of the depth stop 316 to deflect the single wing 304 relative to the main body 102 to interface with the surface 130 of the glenoid 126.

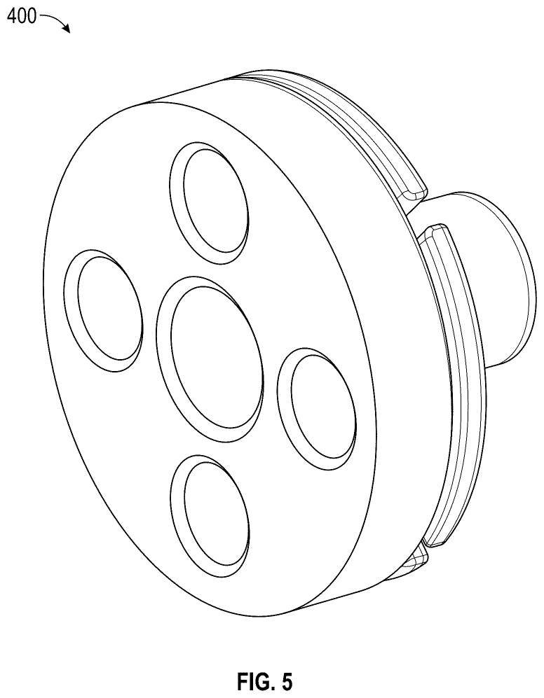

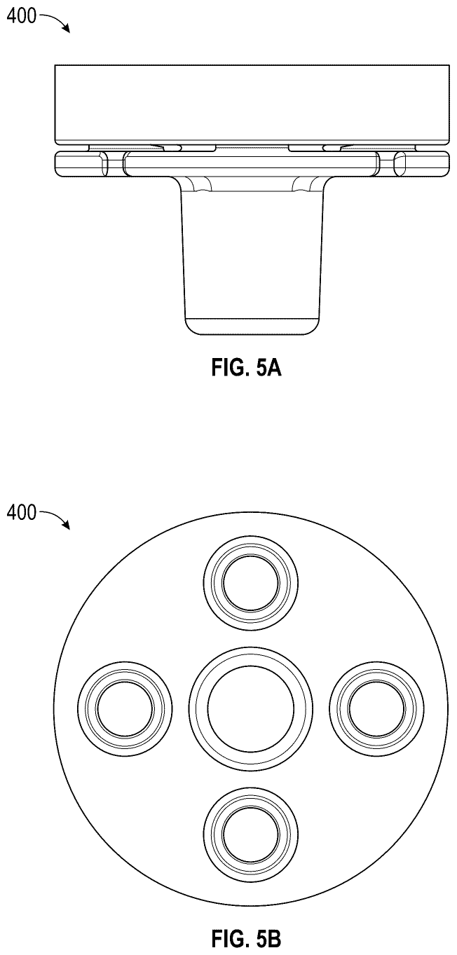

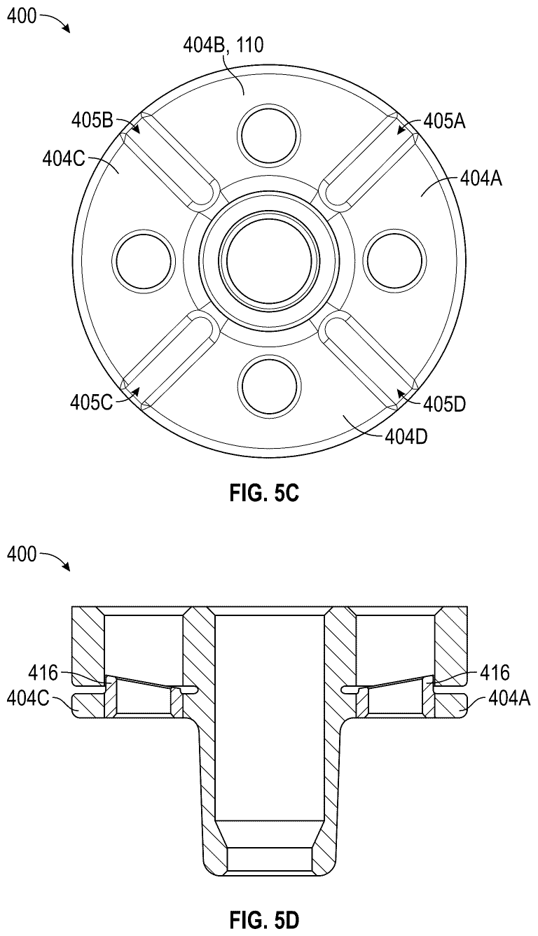

FIGS. 5-5D show an example of a baseplate 400 having a design similar to that discussed previously. However, the baseplate 400 differs in that the baseplate 400 includes four wings 404A, 404B, 404C and 404D (all shown in FIG. 5C only). Together the wings 404A, 404B, 404C and 404D can form substantially all of the distal surface 110 of the baseplate 400 as shown in FIG. 5C. Additionally, the wings 404A, 404B, 404C and 404D can be separated from adjacent ones of the wings 404A, 404B, 404C and 404D by gaps, grooves or recesses 405A, 405B, 405C and 405D, as shown in FIG. 5C.

The wings 404A, 404B, 404C and 404D are moveable in the manner discussed previously to interface with the surface of the glenoid. Put another way, the wings 404A, 404B, 404C and/or 404D can be selectively moved such as by being deflected distally to conform with the surface of the glenoid. As shown in FIG. 5D, the baseplate 400 additionally differs from the baseplate 100 in that the depth stops 416 are separate components from the wings 404A and 404C. The depth stop 416 can be threaded or otherwise connected to wings 404A and 404C (and the wings 404B and 406D although not illustrated in FIG. 5D). As discussed previously, the wings 404A, 404B, 404C and 404D can be selectively deflected independently of one another to desired depths including different depths from one another as needed.

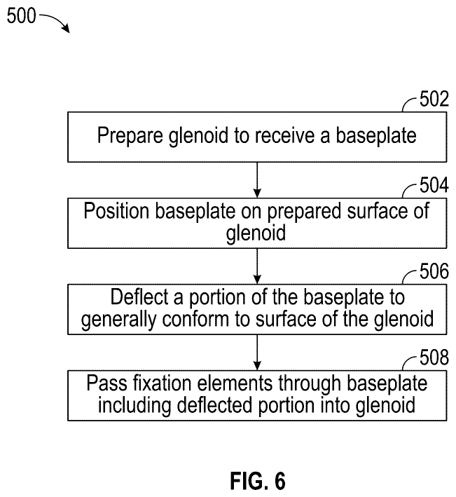

FIG. 6 is a flow diagram of a method 500 of implanting a baseplate to a glenoid during a reverse shoulder arthroplasty. The method 500 can include preparing 502 the glenoid to receive the baseplate. According to another step, the method 500 can include positioning 504 the baseplate on a surface of the glenoid. The method 500 can include deflecting 506 a portion of the baseplate to generally conform with the surface of the glenoid by engaging one or more fixation elements with the portion of the baseplate. Deflecting the portion of the baseplate can include engaging a depth stop with a head of the one or more fixation elements as discussed and shown previously. Deflecting the portion of the baseplate can include selectively deflecting two or more portions of the baseplate (e.g., deflecting two or more wings). The method 500 can include deflecting the two or more portions of the baseplate independently to a different depth from one another.

The method 500 can include passing 508 at least a portion of the one or more fixation elements into the glenoid. The method 500 can further include coupling the depth stop (if used) to the portion of the baseplate sequent to deflecting the portion of the baseplate. The method 500 can include anchoring the baseplate to the glenoid with at least a single fixation element partially received by a centrally positioned peg of the baseplate.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A baseplate configured to mount on a surface of a glenoid and couple with a glenosphere for a reverse shoulder arthroplasty, comprising:
a main body having a plurality of thru holes therein; and
one or more wings coupled to the main body and extending outward therefrom, wherein the one or more wings include at least one thru hole therein, and wherein the one or more wings are configured to be moveable relative to the main body to interface with the surface of the glenoid.

2. The baseplate of claim 1, further comprising a depth stop coupled to the one or more wings, wherein the depth stop includes a thru hole arranged generally coaxial with the at least one thru hole, wherein the depth stop is positioned at least one of adjacent to or at least partially within at least one of the plurality of thru holes of the main body.

3. The baseplate of claim 2, wherein the depth stop is one of: integral with the one or more wings or an insert separate from the one or more wings.

4. The baseplate of claim 3, wherein, when the one or more wings are in an undeflected state, the depth stop includes a proximal surface that is acutely angled relative to a centerline axis of the thru hole.

5. The baseplate of claim 4, wherein the main body has a centerline axis, and wherein an outer radial portion of the depth stop from the centerline axis has an increased depth as compared with an inner radial portion of the depth stop from the centerline axis.

6. The baseplate of claim 5, further comprising one or more fixation elements received by one or more of the plurality of thru holes of the main body and the at least one thru hole of the one or more wings, wherein a head of the one or more fixation elements engages the proximal surface of the depth stop to deflect the one or more wings relative to the main body to interface with the surface of the glenoid.

7. The baseplate of claim 1, further comprising one or more fixation elements received by one or more of the plurality of thru holes of the main body and the at least one thru hole of the one or more wings, wherein the one or more fixation elements are configured to force the one or more wings against the surface of the glenoid.

8. The baseplate of claim 1, wherein the one or more wings include between one and four wings, inclusive, and wherein each of the between one and four wings includes the at least one thru hole.

9. The baseplate of claim 1, further comprising a peg coupled to and extending from the main body, wherein one of the plurality of thru holes of the main body additionally passes through the peg, and wherein the one or more wings are positioned around the peg.

10. The baseplate of claim 9, wherein the peg and the one or more wings are at least partially formed by a porous material configured to facilitate tissue ingrowth.

11. An orthopedic system for a reverse shoulder arthroplasty, comprising:
a one or more fixation elements; and
a baseplate configured to mount on a surface of a glenoid and couple with a glenosphere, the baseplate comprising:
a main body having a plurality of thru holes therein,
one or more wings coupled to the main body and extending outward therefrom,
wherein the one or more wings include at least one thru hole therein, and
a depth stop coupled to each of the one or more wings, wherein the depth stop includes a thru hole with a centerline axis generally aligned with a centerline axis of the at least one thru hole, wherein the depth stop is positioned at least one of adjacent to or at least partially within at least one of the plurality of thru holes of the main body, wherein a proximal surface of the depth stop is configured to be engaged by a head of the one or more fixation elements to deflect the one or more wings relative to the main body to interface with the surface of the glenoid.

12. The orthopedic system of claim 11, wherein the depth stop is one of: integral with the one or more wings or an insert separate from the one or more wings.

13. The orthopedic system of claim 11, wherein, when the one or more wings are in an undeflected state, the depth stop includes a proximal surface that is acutely angled relative to a centerline axis of the thru hole.

14. The orthopedic system of claim 11, wherein the one or more wings include between one and four wings, inclusive, and wherein each of the between one and four wings includes the at least one thru hole.

15. A method of implanting a baseplate to a glenoid during a reverse shoulder arthroplasty, the method comprising:
preparing the glenoid to receive the baseplate;
positioning the baseplate on a surface of the glenoid;
deflecting a portion of the baseplate to generally conform with the surface of the glenoid by engaging one or more fixation elements with the portion of the baseplate; and
passing at least a portion of the one or more fixation elements into the glenoid.

16. The method of claim 15, wherein deflecting the portion of the baseplate includes engaging a depth stop with a head of the one or more fixation elements.

17. The method of claim 16, further comprising coupling the depth stop to the portion of the baseplate sequent to deflecting the portion of the baseplate.

18. The method of claim 15, wherein deflecting the portion of the baseplate includes selectively deflecting two or more portions of the baseplate.

19. The method of claim 18, further comprising deflecting the two or more portions of the baseplate to a different depth from one another.

20. The method of claim 15, further comprising anchoring the baseplate to the glenoid with a single fixation element partially received by a centrally positioned peg of the baseplate.
